(19) United States
(12) Patent Application Publication
(10) Pub. No.: US 2007/0276044 A1
(43) Pub. Date: Nov. 29, 2007

(54) Peeling Cosmetic Method Using a 8-Hexadecene-1,16-Dicarboxylic Acid

(75) Inventors: Isabelle Hansenne, Westfield, NJ (US); Gabrielle Sore, Paris (FR)

Correspondence Address: OBLON, SPIVAK, MCCLELLAND MAIER & NEUSTADT, P.C., 1940 DUKE STREET, ALEXANDRIA, VA, 22314, US

(73) Assignee: L'OREAL, Paris (FR)

(21) Appl. No.: 10/589,975

(22) PCT Filed: Feb. 21, 2005
(86) PCT No.: PCT/FR05/00399

§ 371 Date: Jul. 11, 2007

Related U.S. Application Data
(60) Provisional application No. 60/567,788, filed on May 5, 2004.

(30) Foreign Application Priority Data
Feb. 19, 2004 (FR) 0450307

(51) Int. Cl. A61K 31/194; A61P 17/02

(52) U.S. Cl. 514/574

(57) ABSTRACT

The present invention relates to a cosmetic process for treating visible and/or tactile skin irregularities, comprising the steps consisting in: (a) topically applying to the skin a composition containing, in a physiologically acceptable medium, at least 10% by weight of 8-hexadecene-1,16-dicarboxylic acid, (b) leaving the composition in contact with the skin for a time ranging from 5 minutes to 6 hours, and (c) removing the composition by rinsing. This process in particular makes it possible to attenuate wrinkles and fine lines and/or pigmentary marks and/or scars such as acne or chickenpox marks.

The present invention relates to a cosmetic process for treating visible and/or tactile skin irregularities, which is intended in particular for attenuating wrinkles and/or fine lines and/or pigmentary marks and/or scars such as acne or chickenpox marks, comprising the steps consisting in: (a) topically applying to the skin a composition containing, in a physiologically acceptable medium, at least 10% by weight of 8-hexadecene-1,16-dicarboxylic acid, (b) leaving the composition in contact with the skin for a time ranging from 5 minutes to 6 hours, and (c) removing the composition by rinsing.

8-Hexadecene-1,16-dicarboxylic acid or 9-octadecene-dioic acid is a compound that is predominantly in cis form, obtained by biofermentation of oleic acid in the presence of a mutant yeast of the Candida species. It especially has bleaching and antimicrobial properties, making it possible to envisage its use in deodorant, antidandruff and antiacne products, as described by J. W. Wiechers et al. in Cosmetics & Toiletries, Vol. 117, No. 7, pp. 55-68 (July 2002) and in SÖFW Journal, 128, pp. 2-8 (2002).

It has also been proposed in patent application WO 03/032 941 to use it in compositions for preventing tanning of the skin, in combination with antioxidants. Its use as a depigmenting agent as a replacement for kojic acid or hydroquinone, in particular in cleansing compositions, has also been suggested in document DE-101 50 734.

However, to the Applicant's knowledge, it has never been proposed to use this compound to perform scrubs.

Chemical scrubs are a well known means for improving the surface appearance of the skin, in particular to attenuate pigmentation defects such as actinic lentigo or acne or chickenpox marks, or to smooth out skin texture irregularities, in particular wrinkles and fine lines, by causing limited destruction of the epidermis and of the upper layers of the dermis.

It has already been suggested to use various compounds alone or in combination, and in particular glycolic acid and fruit acids, resorcinol, trichloroacetic acid, phenol and retinoic acid, to perform chemical scrubs. However, although these compounds have given satisfactory results, there nevertheless remains a need for scrubbing compositions that are effective while at the same time being well tolerated.

The Applicant has now found that 8-hexadecene-1,16-dicarboxylic acid can allow effective and well-tolerated chemical scrubs to be performed.

One subject of the present invention is thus a cosmetic process for treating visible and/or tactile skin irregularities, comprising the steps consisting in:

(a) topically applying to the skin a composition containing, in a physiologically acceptable medium, at least 10% by weight of 8-hexadecene-1,16-dicarboxylic acid,

(b) leaving the composition in contact with the skin for a time ranging from 5 minutes to 6 hours, and

(c) removing the composition by rinsing.

The 8-hexadecene-1,16-dicarboxylic acid used according to the invention may be in cis form, in trans form or in a mixture of these two forms. It is especially commercially available from the company Uniqema under the trade name Arlatone Dioic DCA.

The amount of 8-hexadecene-1,16-dicarboxylic acid used according to the invention depends on the desired result and in particular on the depth of scrubbing that it is desired to obtain, which itself depends on the condition of the skin to be improved. To give an order of magnitude, the amount of 8-hexadecene-1,16-dicarboxylic acid may represent from 10% to 50% and preferably from 15% to 35% of the weight of the composition.

The composition used according to the invention is suitable for topical application to the skin and thus generally comprises a physiologically acceptable medium, i.e. a medium that is compatible with the skin and optionally with its integuments.

It may be in any galenical form conventionally used for topical application, provided that it is easily removed by rinsing, and especially in the form of a gel or an aqueous-glycolic or aqueous-alcoholic solution. By adding a fatty or oily phase, it may also be in the form of a dispersion of lotion or emulsion type of liquid or semi-liquid consistency, preferably obtained by dispersing a fatty phase in an aqueous phase (O/W). As a variant, the composition according to the invention may be in the form of a mask. These compositions are prepared according to the usual methods.

When the composition is in emulsion form, the proportion of oily phase of the emulsion may range, for example, from 1% to 30% by weight and preferably from 5% to 20% by weight relative to the total weight of the composition. The oils, emulsifiers and co-emulsifiers used in the composition in emulsion form are chosen from those conventionally used in cosmetics or dermatology. The emulsifier and the co-emulsifier are generally present in the composition in a proportion ranging from 0.3% to 30% by weight and preferably from 0.5% to 20% by weight relative to the total weight of the composition. The emulsion may also contain lipid vesicles.

As fatty substances that may be used in the invention, it is possible to use oils and especially mineral oils (liquid petroleum jelly), oils of plant origin (avocado oil or soybean oil), synthetic oils (perhydrosqualene), silicone oils (cyclomethicone) and fluoro oils (perfluoropolyethers).

As emulsifiers and co-emulsifiers that may be used in the invention, examples that may be mentioned include fatty alkyl ethers of polyalkylene glycols such as oxypropylenated (15 PPG) stearyl ether and oxyethylenated (especially 2 and 21 EO) stearyl ethers.

The composition according to the invention may also contain adjuvants that are common in cosmetics and dermatology, such as thickeners, active agents, preserving agents, solvents and fillers. The amounts of these various adjuvants are those conventionally used in the fields under consideration, for example from 0.01% to 20% of the total weight of the composition. Depending on their nature, these adjuvants may be introduced into the fatty phase or into the aqueous phase. These adjuvants, and the concentrations thereof, should be such that they do not harm the properties of the 8-hexadecene-1,16-dicarboxylic acid.

Thickeners that may be mentioned in particular include: xanthan gum, an optionally crosslinked acrylic acid homopolymer or copolymer, a polyacrylamide, an acrylamidomethylpropanesulfonic acid homopolymer or copolymer, and cellulose derivatives including hydroxypropylcellulose.

The composition advantageously contains at least one compound chosen from ethanol, propylene glycol, dipropylene glycol, isostearic acid, isostearyl alcohol, propylene glycol isostearate, glyceryl isostearate and dimethyl isosorbide, which improve the solubility of the 8-hexadecene-1,16-dicarboxylic acid.

In addition, according to one preferred embodiment, the composition used according to the invention contains at least one β-hydroxy acid.

Thus, this composition may comprise, in a physiologically acceptable medium suitable for topical application to the skin: (a) from 10% to 50% by weight of 8-hexadecene-1,16-dicarboxylic acid and (b) from 0.1% to 15% by weight and preferably from 0.2% to 10% by weight of at least one β-hydroxy acid, relative to the total weight of the composition.

β-Hydroxy acids that may be mentioned include salicylic acid and derivatives thereof, in particular the compounds of formula (I) below, or a salt of such a derivative:

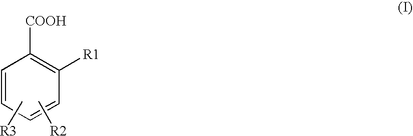

in which:

R₁ represents a hydroxyl radical or an ester of formula:
—O—CO—R₄

in which R₄ is a saturated or unsaturated aliphatic radical containing from 1 to 26 carbon atoms and preferably from 1 to 18 carbon atoms, an amine or thiol function optionally substituted with an alkyl radical containing from 1 to 18 carbon atoms and preferably from 1 to 12 carbon atoms,

R₂ and R₃, independently of each other, are in position 3, 4, 5 or 6 on the benzene nucleus and represent, independently of each other, a hydrogen atom or a radical:
—(O)ₙ—(CO)ₘ—R₅

in which n and m, independently of each other, are each an integer equal to 0 or 1; on condition that R₂ and R₃ are not simultaneously hydrogen atoms;

R₅ represents a hydrogen atom, a linear, branched or cyclized saturated aliphatic radical containing from 1 to 18 carbon atoms, an unsaturated radical containing from 3 to 18 carbon atoms, bearing one to nine conjugated or non-conjugated double bonds, the radicals possibly being substituted with at least one substituent chosen from halogen atoms (fluorine, chlorine, bromine or iodine), trifluoromethyl radicals, hydroxyl in free form or esterified with an acid containing from 1 to 6 carbon atoms, or carboxyl in free form or esterified with a lower alcohol containing from 1 to 6 carbon atoms.

The salicylic acid derivative of formula (I) is preferably such that R₁ represents a hydroxyl radical, R₂ represents a hydrogen atom, R₃ is in position 5 of the benzene nucleus and represents a radical —CO—R₅ in which R₅ represents a saturated aliphatic radical containing from 3 to 15 carbon atoms.

According to one preferred embodiment of the invention, the salicylic acid derivative of formula (I) is chosen from 5-n-octanoylsalicylic acid, 5-n-decanoylsalicylic acid, 5-n-dodecanoylsalicylic acid, 5-n-octylsalicylic acid, 5-n-heptyloxysalicylic acid, 4-n-heptyloxysalicylic acid, 5-tert-octylsalicylic acid, 3-tert-butyl-5-methylsalicylic acid, 3-tert-butyl-6-methylsalicylic acid, 3,5-diisopropylsalicylic acid, 5-butoxysalicylic acid, 5-octyloxysalicylic acid, 5-propanoylsalicylic acid, 5-n-hexadecanoylsalicylic acid, 5-n-oleoylsalicylic acid, 5-benzoylsalicylic acid, monovalent and divalent salts thereof, and mixtures thereof. It is more particularly 5-n-octanoyl-salicylic acid (INCI name: Capryloyl salicylic Acid).

The amount of β-hydroxy acid may represent from 0.1% to 15% and preferably from 0.2% to 10% relative to the total weight of the composition.

As a variant or in addition, the composition used according to the invention may comprise at least one desquamating agent chosen especially from: α-hydroxy acids such as glycolic acid, citric acid, lactic acid, tartaric acid, malic acid or mandelic acid; ascorbic acid and derivatives thereof such as ascorbyl glucoside and magnesium ascorbyl phosphate; nicotinamide; urea; and (N-2-hydroxyethylpiperazine-N-2-ethane)sulfonic acid (HEPES).

As indicated previously, the process according to the invention is intended to be used to perform superficial chemical scrubbing aimed at attenuating visible and/or tactile skin irregularities, and in particular at attenuating wrinkles and fine lines and/or pigmentary marks and/or scars. It is thus preferably performed on individuals with acne and/or wrinkles and/or scars and/or pigmentation defects such as melasmas and senile or actinic lentigo.

This process may especially be performed by a beautician.

According to this process, the composition used according to the invention may be applied to the face and/or the neck and/or the neckline and/or the hands and/or the back by any means allowing uniform distribution and especially using cottonwool, a cotton bud, a brush, a gauze, a spatula or a pad, or alternatively by spraying, and may be removed by rinsing with water or with a mild detergent after remaining in contact with the skin for a time of between 5 minutes and 6 hours and preferably between 5 minutes and 30 minutes.

To optimize its effects, the scrubbing process preferably comprises additional steps of preparing the skin for the scrub (to improve the efficacy and homogeneity of the scrub) and/or of skincare after scrubbing using compositions containing smaller amounts of 8-hexadecene-1,16-dicarboxylic acid than the composition described above.

The compositions used in these preliminary and additional steps may be applied morning and evening, for example, optionally in combination with a composition intended to protect the skin against the effects of UV. The pretreatment composition may be applied for one to four weeks, and the post-treatment composition for one day to eight weeks, for example.

The above scrubbing process, including the optional preliminary and additional steps, may be performed once only or repeated up to five times, if necessary. The frequency of the application is between twice a week and once every three weeks. It is preferably one application per week.

The invention will now be illustrated by the non-limiting examples that follow. In these examples, the amounts are indicated as weight percentages.

### EXAMPLES

### Example 1

### In vivo Evaluation of the Effect of 8-Hexadecene-1,16-Dicarboxylic Acid

Protocol

The efficacy of 8-hexadecene-1,16-dicarboxylic acid was evaluated on a panel of 12 Caucasian women from 55 to 65 years old, showing signs of photo-ageing on the forearms.

To do this, a composition A containing 10% 8-hexa-decene-1,16-dicarboxylic acid was applied to a 9 cm² area located on the upper face of the forearms of the individuals, at a rate of 15 mg/cm². The solution was left for 5 minutes on the application area, and then removed using a compress soaked in warm water. The treatment was performed once a day for five consecutive days.

An evaluation of the tolerance (irritation and subjective sensations) was performed after each application. Non-invasive measurements also allowed the transepidermal water loss (TWL), the colour of the skin (L*a*b* system), the skin elasticity (by cutometry) and the skin microrelief (by taking impressions) to be measured, at Tₒ and after 8 days.

The TWL was measured, precisely, using a Tewameter (Courage and Khazaka), the measurement of the rate of cutaneous evaporation, expressed in g/m²/h being taken after stabilization of the value, i.e. about one minute after placing the probe on the surface of skin to be analysed. The calorimetric measurements were taken using a CR300 chromameter (Minolta) and then transferred to a computer of PC type. The elasticity measurements were taken using a Cutometer (Courage and Khazaka). The impressions were made using a Silflo elastomeric mastic followed by an image analysis system intended to measure: the mean area of the hollows, their mean length, their mean depth and their form factor.

Composition A was compared with a composition B, applied under the same conditions (to another site of the forearm of each of the individuals) and containing 30% glycolic acid in 60% water and 10% glycerol, used as reference composition). After applying this composition, the area of treated skin was, however, neutralized and rinsed to soothe the stinging.

An untreated area of skin of the forearm was used as control for each individual.

Compositions A and B were compared by means of a Student test for paired data. The significance level of the tests was set at p<0.05.

Results

The results of the TWL test are collated in the table below:

| | | TWL on the | | Variation |
|---|---|---|---|---|
| | TWL at Tₒ | 8^{th} day | Difference | relative to |
| Composition | (g/m²/h) | (g/m²/h) | (D8 − D1) | the control |
| A | 9.35 | 9.46 | 0.11 | 0.91 |
| B | 9.45 | 8.78 | −0.67 | 0.14 |
| Control | 9.45 | 8.65 | −0.81 | 0.00 |
| site | | | | |

As emerges from this table, the application of composition A containing 6-hexadecene-1,16-dicarboxylic acid resulted in an increase in the transepidermal water loss from the skin, greater than that observed with glycolic acid, which is a reference scrubbing active agent. These results suggest that this compound is a good candidate as a scrubbing active agent.

### Example 2

### Anti-Wrinkle Scrubbing Composition

The composition below is prepared in the usual manner for those skilled in the art.

| | |
|---|---|
| 8-Hexadecene-1,16-dicarboxylic acid | 15% |
| Mandelic acid | 10% |
| Water | 20% |
| Polyethylene glycol | qs 100% |

This composition may be applied in the form of a scrub to attenuate facial wrinkles and fine lines.

### Example 3

### Bleaching Scrubbing Composition

The composition below is prepared in the usual manner for those skilled in the art.

| | |
|---|---|
| 8-Hexadecene-1,16-dicarboxylic acid | 15% |
| Calcium D-pantetheine sulfonate | 1% |
| Water | 15% |
| Ethanol | qs 100% |

This composition may be applied in the form of a scrub to attenuate dyschromias (actinic or senile lentigo, and melasmas).

### Example 4

### Anti-Acne Scrubbing Composition

The composition below is prepared in the usual manner for those skilled in the art.

| | |
|---|---|
| 8-Hexadecene-1,16-dicarboxylic acid | 40% |
| 5-n-Octanoylsalicylic acid | 2% |
| Ethanol | qs 100% |

This composition may be applied in the form of a scrub to attenuate acne marks.

1. A cosmetic process for treating at least one skin irregularity selected from the group consisting of a visible skin irregularity, a tactile skin irregularity, and a combination thereof, comprising
(a) topically applying to the skin a composition comprising, in a physiologically acceptable medium, at least 10% by weight of 8-hexadecene-1,16-dicarboxylic acid,
(b) leaving the composition in contact with the skin for a time ranging from 5 minutes to 6 hours, and
(c) removing the composition by rinsing.

2. The process according to claim 1, wherein the composition is left in contact with the skin, in (b), for a time ranging from 5 minutes to 30 minutes.

3. The process of claim 1, wherein the at least one skin irregularity is selected from the group consisting of at least one wrinkle, at least one fine line, at least one pigmentary mark, at least one scar, and combinations thereof.

4. The process of claim 3, wherein the at least one skin irregularity is the at least one scar, and wherein the at least one scar is an acne or a chickenpox mark.

5. The process of claim 1, wherein the process is performed on a human with acne, at least one wrinkle, at least one scar, at least one pigmentation defect, or a combination thereof.

6. The process of claim 1, wherein the amount of 8-hexadecene-1,16-dicarboxylic acid represents from 10% to 50% of the total weight of the composition.

7. The process of claim 6; wherein the amount of 8-hexadecene-1,16-dicarboxylic acid represents from 15% to 35% of the total weight of the composition.

8. The process of claim 1, wherein the composition comprises, in a physiologically acceptable medium suitable for topical application to the skin: from 10% to 50% by weight of 8-hexadecene-1,16-dicarboxylic acid and from 0.1% to 15% by weight of at least one β-hydroxy acid, relative to the total weight of the composition.

9. The process according to claim 8, wherein the at least one β-hydroxy acid is selected from the group consisting of a salicylic acid, a salt of a salicyclic acid, and combinations thereof.

10. The process of claim 9, wherein the at least one β-hydroxy acid is a salicyclic acid, and wherein the salicylic acid is 5-n-octanoylsalicylic acid.

11. The process of claim 8, wherein the amount of the at least one β-hydroxy acid represents from 0.2% to 10% relative to the total weight of the composition.

12. The process of claim 2, wherein the at least one skin irregularity is selected from the group consisting of at least one wrinkle, at least one fine line, at least one pigmentary mark, at least one scar, and combinations thereof.

13. The process of claim 12, wherein the at least one skin irregularity is the at least one scar, and wherein the at least one scar is an acne or a chickenpox mark.

14. The process of claim 2 wherein the composition comprises, in a physiologically acceptable medium suitable for topical application to the skin: from 10% to 50% by weight of 8-hexadecene-1,16-dicarboxylic acid and from 0.1% to 15% by weight of at least one β-hydroxy acid, relative to the total weight of the composition.

15. The process according to claim 14, wherein the at least one β-hydroxy acid is selected from the group consisting of a salicylic acid, a salt of a salicyclic acid, and combinations thereof.

16. The process of claim 15, wherein the at least one β-hydroxy acid is a salicyclic acid, and wherein the salicylic acid is 5-n-octanoylsalicylic acid.

17. The process of claim 14, wherein the amount of the at least one β-hydroxy acid represents from 0.2% to 10% relative to the total weight of the composition.

18. The process of claim 3 wherein the composition comprises, in a physiologically acceptable medium suitable for topical application to the skin: from 10% to 50% by weight of 8-hexadecene-1,16-dicarboxylic acid and from 0.1% to 15% by weight of at least one β-hydroxy acid, relative to the total weight of the composition.

19. The process according to claim 18, wherein the at least one β-hydroxy acid is selected from the group consisting of a salicylic acid, a salt of a salicyclic acid, and combinations thereof.

20. The process of claim 19, wherein the at least one β-hydroxy acid is a salicyclic acid, and wherein the salicylic acid is 5-n-octanoylsalicylic acid.
